(19) United States
(12) Patent Application Publication
(10) Pub. No.: US 2014/0127182 A1
(43) Pub. Date: May 8, 2014

(54) Using GNMT as a Novel Therapeutic or Preventing Agent for Fatty Liver Related Diseases

(71) Applicant: NATIONAL YANG-MING UNIVERSITY, Taipei City (TW)

(72) Inventors: Yi-Ming CHEN, Taipei (TW); Chia-Hung YEN, Taipei (TW); Yi-Jen LIAO, Taipei (TW); Kuan-Hsuan CHEN, Taipei (TW)

(73) Assignee: National Yang-Ming University, Taipei City (TW)

(21) Appl. No.: 13/890,742

(22) Filed: May 9, 2013

(30) Foreign Application Priority Data
Nov. 8, 2012 (TW) 101141570

(51) Int. Cl. A61K 38/45; G01N 33/573

(52) U.S. Cl. CPC A61K 38/45; G01N 33/573
USPC 424/94.5; 435/7.4

(57) ABSTRACT

The present invention relates to a use of Glycine N-methyltransferase (GNMT) in treating or preventing fatty liver related disease, such as nonalcoholic fatty liver disease (NAFLD) or hepatocellular carcinoma (HCC). The use of GNMT for treating or preventing fatty liver diseases is achieved by enhancing GNMT-NPC2 interaction, and thus decreasing or preventing the accumulation of lipid and cholesterol in hepatic cells or tissues.

### FIELD OF THE INVENTION

The present invention relates to a method of using Glycine N-methyltransferase (GNMT) to treat or prevent fatty liver related diseases including nonalcoholic fatty liver disease (NAFLD) and hepatocellular carcinoma (HCC). In particular, the use of GNMT for treating or preventing fatty liver diseases is achieved by enhancing GNMT-NPC2 interaction, and thus decreasing or preventing the accumulation of lipid and cholesterol in hepatic cells or tissues.

### BACKGROUND OF THE INVENTION

Glycine N-methyltransferase (GNMT) regulates the ratio of S-adenosylmethionine(SAM) to S-adenosylhomocysteine (SAH) by catalyzing sarcosine synthesis from glycine (Kerr S J. J Biol Chem 1972; 247: 4248-4252). In addition to folate, GNMT binds polyaromatic hydrocarbons (PAHs) and aflatoxins (Yeo E J, et al. Proc Natl Acad Sci U S A 1994; 91: 210-214). We previously described diminished GNMT expression levels in both human HCC cell lines and tumor tissues (Liu H H, et al. J Biomed Sci 2003; 10: 87-97). Results from genotypic analyses of several human GNMT gene polymorphisms revealed a loss of heterozygosity in 36-47% of tumor tissues collected from HCC patients.

Fatty liver, also known as fatty liver disease (FLD), is a reversible condition where large vacuoles of triglyceride fat accumulate in liver cells via the process of steatosis (i.e. abnormal retention of lipids within a cell). Despite having multiple causes, fatty liver can be considered a single disease that occurs worldwide in those with excessive alcohol intake and those who are obese (with or without effects of insulin resistance). The condition is also associated with other diseases that influence fat metabolism (Reddy J K, Rao M S, Am. J. Physiol. Gastrointest. Liver Physiol. 2006; 290 (5): G852-8). Morphologically, it is difficult to distinguish alcoholic FLD from nonalcoholic FLD, and both Show microvesicular and macrovesicular fatty changes at different stages.

Non-alcoholic fatty liver diseases (NAFLD) include illnesses ranging from hepatic steatosis to intermediate lesions, non-alcoholic steatohepatitis (NASH), and cirrhosis. Epidemiologists estimate that between 20% and 30% of all adults living in the United States and other developed countries have some form of NAFLD. NAFLD is considered the hepatic event in the metabolic syndrome and is associated with hepatocellular carcinoma (HCC) development. The liver plays a critical role in whole-body lipid metabolism. Deterioration in lipid uptake, transport, excretion, synthesis, and catabolic mechanisms serves as the basis for NAFLD development. It is important to note that cholesterol content is increased in human fatty liver tissues, suggesting that cholesterol metabolism is deregulated in NAFLD. However, the reason for the cholesterol accumulation is still unclear.

Niemann-Pick Type C2 (NPC2) protein is a small soluble glycoprotein and mainly expresses in liver, kidney and testis. NPC2 plays an important role in regulating intracellular cholesterol trafficking and homeostasis through direct binding with free cholesterol. Deficiency of NPC2 in mice results in cholesterol accumulation in the liver tissues. Glycine N-methyltransferase (GNMT) is abundantly expressed in the liver cytosol, but is down-regulated in human HCC tissues. Previously, we generated a GNMT knockout (Gnmt−/−) mice and reported on their tendencies toward chronic hepatitis, glycogen storage (U.S. Pat. No. 7,759,542).

Based on the demonstration that GNMT regulates the homeostasis of cholesterol. metabolism, and hepatic cholesterol accumulation may result from downregulation of GNMT and instability of its interactive protein NPC2. Novel therapeutics for fatty liver related diseases, such as Non-alcoholic fatty liver diseases, steatohepatitis, and HCC may be developed by using this concept.

### SUMMARY OF THE INVENTION

This invention is based on the unexpected discovery that Gnmt−/− mice impaired cholesterol metabolism and developed steatohepatitis. In this invention, we used full-length human GNMT as bait in a yeast two-hybrid screen system and identified NPC2 as a GNMT-interactive protein. Accordingly, this study aimed to investigate the role of GNMT-NPC2 interaction in the regulation of hepatic cholesterol homeostasis.

Therefore, in one aspect of the present invention, it is provided a pharmaceutical composition for in treating or preventing fatty liver related disease, comprising Glycine N-methyltransferase (GNMT) protein as active ingredient, and a pharmaceutically acceptable carrier, diluent or excipient.

In one embodiment of this invention, the GNMT protein is used to enhance the activity and stability of NPC2 protein in liver cells. In certain embodiments, the pharmaceutical composition of this invention is used to improve GNMT-NPC2 interaction, and further to decrease or prevent cholesterol accumulation in liver cells.

The term “prophylactic” or “therapeutic” treatment is art-recognized and refers to administration of a drug to a host. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

GNMT protein can be formulated for a variety of modes of administration, including systemic and topical or localized administration. Techniques and formulations generally may be found in Remington's Pharmaceutical Sciences, Meade Publishing Co., Easton, Pa. For parenteral administration, injection is preferred, including intramuscular, intravenous, intraperitoneal, and subcutaneous. For injection, the compounds can be formulated in liquid solutions, preferably in physiologically compatible buffers such as Hank's solution or Ringer's solution. In addition, the compounds may be formulated in solid form and re-dissolved or suspended immediately prior to use. Lyophilized forms are also included.

For oral administration, the compositions can be formulated by combining the active compounds with pharmaceutically acceptable carriers well known in the art. Such carriers enable the compositions of the invention to be formulated as tablets, pills, lozenges, dragees, capsules, liquids, gels, syrups, slurries, suspensions and the like, for oral ingestion by a patient. Pharmaceutical preparations for oral use can be made using a solid excipient, optionally grinding the resulting mixture, and processing the mixture of granules, after adding other suitable auxiliaries if desired, to obtain tablets or dragee cores. Useful excipients are, in particular, fillers such as sugars, including lactose, sucrose, mannitol, or sorbitol, cellulose preparations such as, for example, maize starch, wheat starch, rice starch and potato starch and other materials such as gelatin, gum tragacanth, methyl cellulose, hydroxypropylmethyl-cellulose, sodium carboxymethylcellulose, and/or polyvinyl-pyrrolidone (PVP). If desired, disintegrating agents may be added, such as cross-linked polyvinyl pyrrolidone, agar, or alginic acid. A salt such as a sodium alginate may also be used.

In another aspect, this invention features a method for diagnosing the occurrence of fatty liver disease in a tested animal, comprising collecting a blood or liver sample from the animal; and detecting the content or expression level of GNMT protein by using a detecting agent.

In certain embodiments, the detecting agent for GNMT protein comprises an anti-GNMT antibody. The term “antibody” herein is used in the broadest sense and specifically includes full-length monoclonal antibodies, polyclonal antibodies, multispecific antibodies (e.g., bispecific antibodies), and antibody fragments, as long as they exhibit the desired biological activity.

Other features or advantages of the present invention will be apparent from the following drawings and detailed description of several embodiments, and also from the appending claims.

### BRIEF DESCRIPTION OF THE DRAWINGS

The patent or application file contains at least one drawing executed in color. Copies of this patent or patent application publication with color drawing(s) will be provided by the Office a upon request and payment of the necessary fee.

FIGS. 1A-1D show that Gnmt^{-/-} mice developed steatohepatitis and hyperlipidemia. FIG. 1A is Hematoxylin and eosin staining of liver tissues from WT and Gnmt^{-/-} mice at 11 wks and 9 months of age. Original magnification 200×. Inset: Higher magnification image of microvesicular intracytoplasmic lipid droplet. FIG. 1B is Filipin and Nile red staining of liver tissues from 11-wk-old WT and Gnmt^{-/-} mice. Arrow, free cholesterol; arrowhead, hydrophobic lipids. Scale bar, 20 μm. Hepatic (C) and serum cholesterol (D), free cholesterol and LDL levels in 11-wk-old WT and Gnmt^{-/-} mice (n=6 mice per group). *P<0.05; **P<0.01.

FIGS. 2A-2D show that cholesterol metabolism is impaired in the liver tissues of Gnmt^{-/-} mice. FIG. 2A and 2B are analyses of genes involved in hepatic cholesterol uptake (ABCA1 and SR-B1), cholesterol synthesis (HMGCR and SREBP2), cholesterol trafficking (NPC1, NPC2 and StAR) and hepatic cholesterol efflux (ABCG1) in 11-wk-old WT and Gnmt^{-/-} mice (n=5-6 mice per group) by Western blot (A) and real-time PCR (B), respectively. The is expression of each gene is normalized to GAPDH. *P<0.05; **P<0.01. FIG. 2C shows ¹³¹I-labeled 6β-iodocholesterol injection site. FIG. 2D is the representative SPECT images of WT and Gnmt^{-/-} mice after injection of ¹³¹I-labeled 6β-iodocholesterol (n=2 in each group).

FIGS. 3A-F are diagrams showing the interaction and colocalization of GNMT with NPC2 in cytosol. 293T cells were cotransfected with GNMT-Flag and NPC2-HA for 24 h and is harvested for coimmunoprecipitation. In FIG. 3A, GNMT was precipitated by anti-Flag beads and immunoblotted with anti-HA to detect NPC2 expression (arrowhead). NPC2 was precipitated by anti-HA beads and immunoblotted with anti-Flag to detect GNMT expression (arrowhead). In FIG. 3B, SK-Hep1 cells were transfected with GNMT-Flag for 24 h and harvested for coimmunoprecipitation. GNMT was precipitated by anti-Flag beads and immunoblotted with anti-NPC2 antibody to detect endogenous NPC2 expression (arrowhead). In FIG. 3C, liver lysates from WT mice were precipitated with anti-GNMT antibodies and immunoblotted with anti-NPC2 antibodies to detect endogenous NPC2 expression (arrowhead). In FIG. 3D, Cytosol and lysosome fractions were isolated from the liver tissues of WT mice. Western blot analysis was used to detect subcellular NPC2 and GNMT localization. LAMP1 and cathepsin D are indicated as lysosome markers; α-tubulin is indicated as a cytosol marker. In FIG. 3E, liver cytosol fractions from WT mice were precipitated with anti-GNMT antibodies and immunoblotted with anti-NPC2 antibodies to detect endogenous NPC2 expression (arrowhead). In FIG. 3F, HuH7 cells were transfected with GNMT-Flag and fixed for immunofluorescence assays using antibodies against Flag, NPC2 and Lysotracker to detect colocalization among GNMT, NPC2 and lysosomes. Colocalization percentage is shown in each merge panel. Scale bar, 20 μm. IB, immunoblot.

FIGS. 4A-B show that GNMT stabilizes NPC2 protein. FIG. 4A is the analysis of NPC2 degradation followed by cycloheximide (CHX) treatment. The 293T cells were transfected with either NPC2-HA or GNMT-Flag plus NPC2-HA for 24 h before treatment with CHX for the indicated hours. NPC2 degradation was analyzed using Western blot. Single arrowheads indicate mono-glycosylated NPC2; double arrowheads indicate diglycosylated NPC2. FIG. 4B is the Pulse-chase analysis of NPC2. The 293T cells were transfected with either NPC2-HA or GNMT-Flag plus NPC2-HA for 24 h; ³⁵S-Met/Cys incorporation measurements were used to determine NPC2 half-lives.

FIGS. 5A-D show the colocalization of GNMT with NPC2 and regulating hepatic cholesterol homeostasis following LDL and progesterone treatment, CHO cells were cultured in normal medium (1) and transfected with either NPC2-DsRed (in FIG. 5A) or GNMT-GFP (in FIG. 5B) and NPC24)sRed for 24 hrs, After reseeding, CHO cells were cultured in LPDS (2), LDL (3), or LDL plus progesterone (4) medium for 24 hrs. After washing out progesterone at 6, 12, 18, or 24 hrs (5-8), confocal microscopy was used to detect colocalization between NPC2-DsRed (red) and GNMT-GFP (green). NPC2-GNMT colocalization percentages are shown in panel B. In FIG. 5C, SK-Hep1 cells were infected with Ad-GNMT (50 MOI) for 24 hrs, followed by treatment with LDL or LDL plus progesterone for 24 hrs. After washing out progesterone at 6 or 18 hrs, cells were harvested for cytosol-lysosome fractionation experiments. NPC2 and GNMT expression were analyzed using western blot assays. LAMP1 and αtubulin are indicated as lysosome and cytosol markers, respectively. In FIG. 5D, Ad-GNMT-infected and Ad-vector-infected SK-Hep1 cells (50 MOI) were cultured in normal and LPDS media, followed by treatment with LDL or LDL plus progesterone for 24 hrs. After washing out progesterone at 6 or 18 hrs, cells were harvested for lipid extraction to determine cholesterol concentrations. Data represent mean±SD. *, P<0.05.

FIGS. 6A-E show that GNMT is downregulated in fatty liver tissues by the Western blot analyses of hepatic GNMT expression from WT mice fed with a high-cholesterol diet (in FIG. 6A), high-fat diet (in FIG. 6B) or MCD diet (in FIG. 6C), but not in ob/ob mice (FIGS. 6D-6E).

FIGS. 7A-D show that NPC2 is dysregulated in fatty liver tissues by the Western blot analysis of NPC2 expression from WT mice fed with high-cholesterol diet (in FIG. 7A), high-fat diet (in FIG. 7B) or MCD diet (in FIG. 7D). FIG. 7C shows NPC2 expression level in 8-wk-old ob/ob mice (n=3-5 per group) compared to WT mice.

### DETAILED DESCRIPTION OF THE INVENTION

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent” which is a biologically, physiologically, or pharmacologically active substance (or substances) that acts locally or systemically in a subject.

In certain embodiments, the method of using Glycine N-methyltransferase (GNMT) to treat or prevent fatty liver related diseases may comprises administrating GNMT protein to an animal having need of the treatment. The animal may include human and other mammals.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety. Further, any mechanism proposed below does not in any way restrict the scope of the claimed invention.

### EXAMPLES

### Example 1

### Gnmt−/− Mice Develop Hepatic Steatosis And Hyperlipidemia

Firstly, we used a GNMT knock-out mouse which showed abnormal liver function and suffered from glycogen storage disease (Gnmt^{-/-} mouse model, disclosed in U.S. Pat. No. 7,759,542) to investigate the role of GNMT in lipid metabolism, specifically comparing hepatic steatosis and serum lipid levels between WT and Gnmt^{-/-} mice.

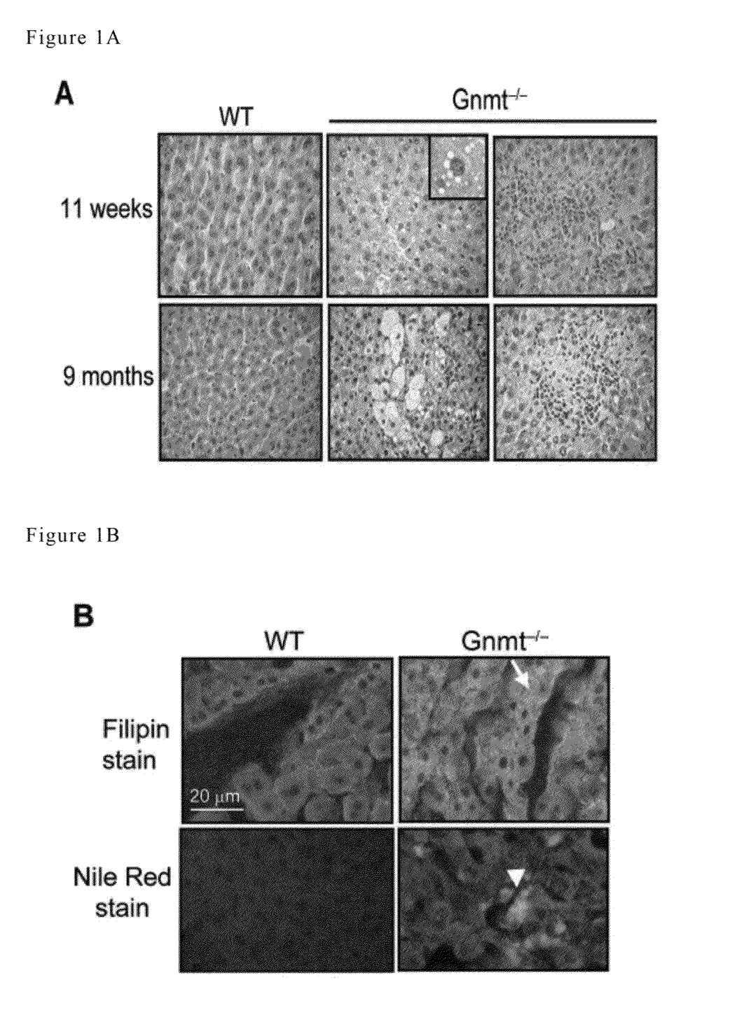

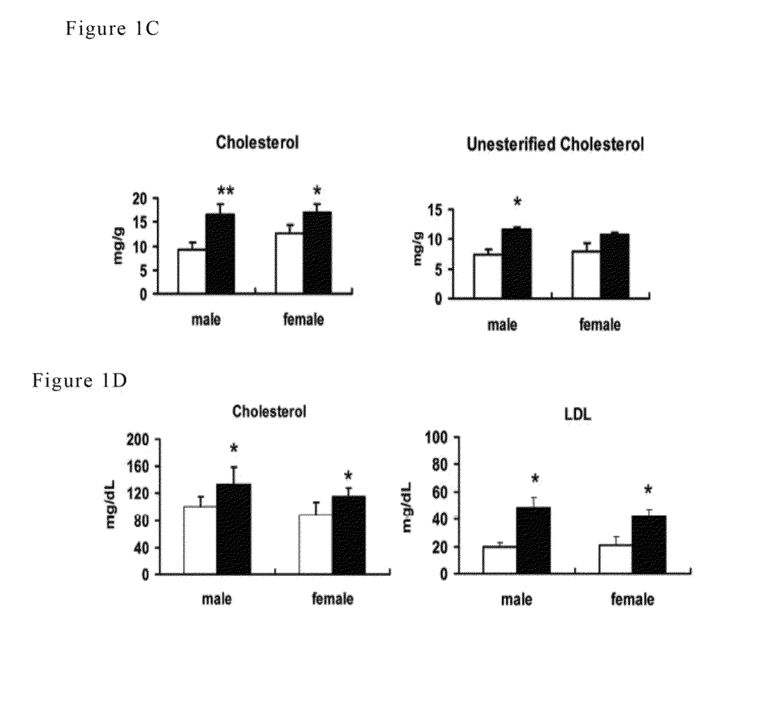

As shown in FIG. 1, liver sections taken from Gnmt^{-/-} mice at 11 wks and 9 months of age showed macrovesicular and microvesicular intra-cytoplasmic lipid droplets and inflammatory infiltration (FIG. 1A). Results from filipin fluorescence and Nile red staining indicate accumulations of free cholesterol and neutral lipids in Gnmt^{-/-} mice livers (FIG. 1B). In addition, analyses of total lipids in mouse liver tissues and serum samples showed that both male and female Gnmt^{-/-} mice had significantly higher cholesterol and LDL levels than WT mice (FIGS. 1C, D; P<0.05). Combined, the data suggest that GNMT loss results in hepatic steatosis and hyperlipidemia.

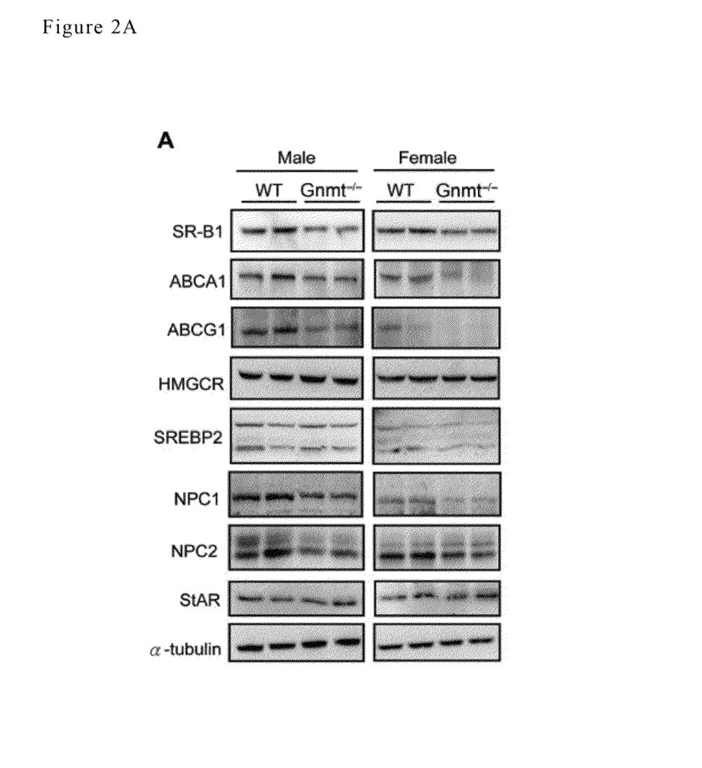

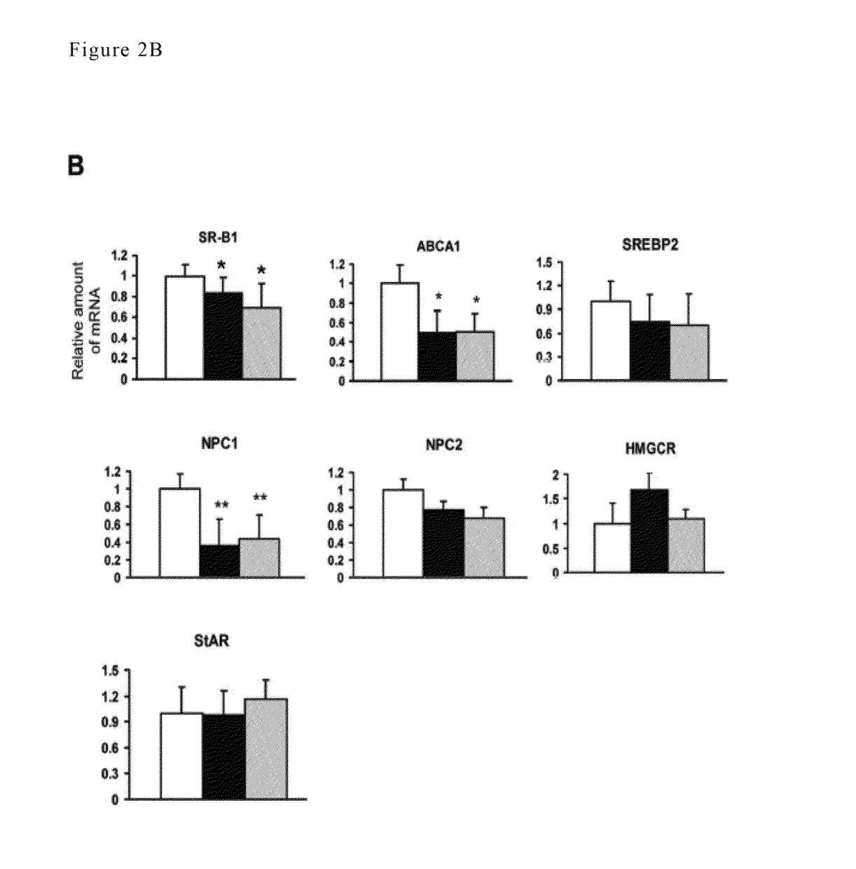

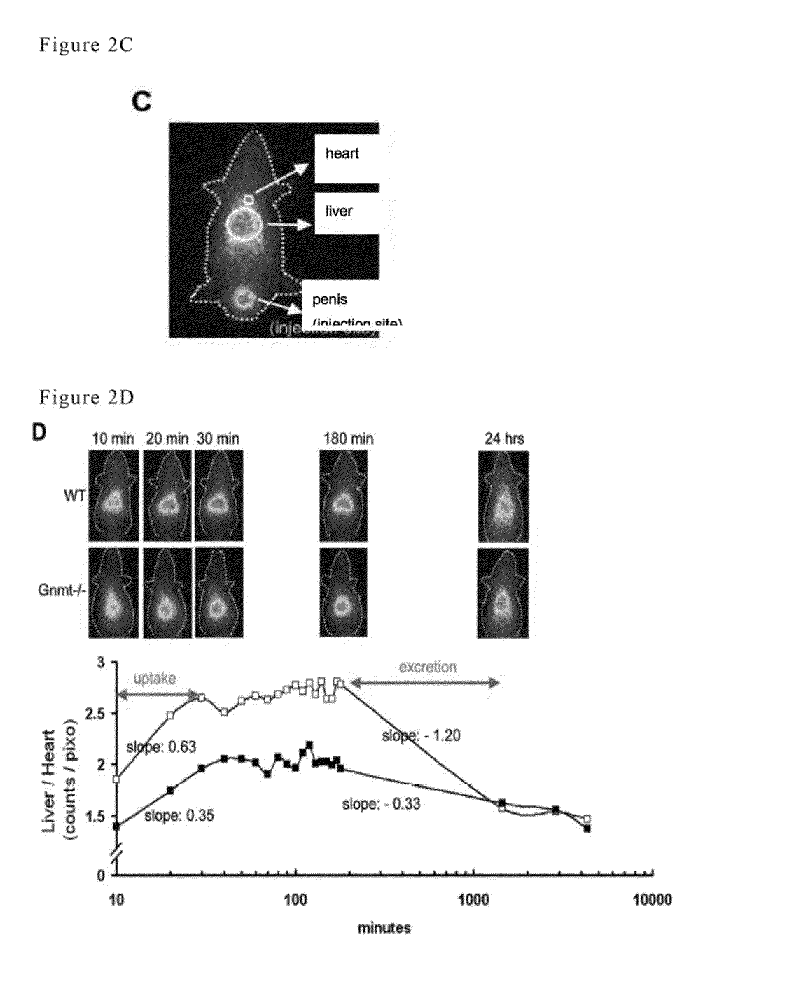

To delineate the molecular mechanisms responsible for hepatic cholesterol accumulation in Gnmt^{-/-} mice, we analyzed protein and mRNA expression levels for the following genes during different stages of cholesterol metabolism: (a) hepatic cholesterol uptake: ABCA1 and SR-B1; (b) cholesterol synthesis: HMGCR and SREBP2; (c) cholesterol trafficking: NPC1, NPC2 and StAR; and (d) hepatic cholesterol efflux: ABCG1. As shown in FIGS. 2A and 2B, proteins involved in cholesterol uptake (SR-B1 and ABCA1), efflux (ABCG1) and trafficking (NPC1) were significantly downregulated in both genders of Gnmt^{-/-} mice compared with WT mice (P<0.05). Although NPC2 protein levels were significantly downregulated in Gnmt mice, no significant changes were noted in mRNA levels. There were no differences in HMGCR. SREBP2 and StAR expression between WT and Gnmt^{-/-} mice.

To further evaluate and compare cholesterol uptake and excretion between WT and Gnmt mice, we injected ¹³¹I-labeled 6β-iodocholesterol into mouse penis and normalized liver single photon emission computed tomography (SPECT) images to heart SPECT images at different time points (FIG. 2C). Compared to WT mice, Gnmt mice expressed much lower radioactivity and slower cholesterol uptake rates in their livers 10-30 min after injection (slope=0.63 versus slope=0.35) (FIG. 2D). The excretion of 131I-labeled 6β-iodocholesterol from the liver was detected at 3 and 24 h after injection. Gnmt^{-/-} mice excreted cholesterol at a much slower rate than WT mice (slope=−0.3 versus slope=−1.2) (see FIG. 2D).

### Example 2

### GNMT-NPC2 Interaction Influences Intracellular Cholesterol Homeostasis

Identification of NPC2 as a GNMT interacting protein

To identify proteins that interact with GNMT, we used full-length human GNMT as bait in a yeast two-hybrid screen system for a human liver cDNA library. After three rounds of screening, we selected a cDNA clone containing a nucleotide sequence encoding amino acid residues 43-151 of the NPC2 protein for further study. Specific GNMT-NPC2 interaction and colocalization were analyzed by coimmunoprecipitation, confocal microscopic examination and a combination of lysosomal/cytosol fractionation and Western blot analysis.

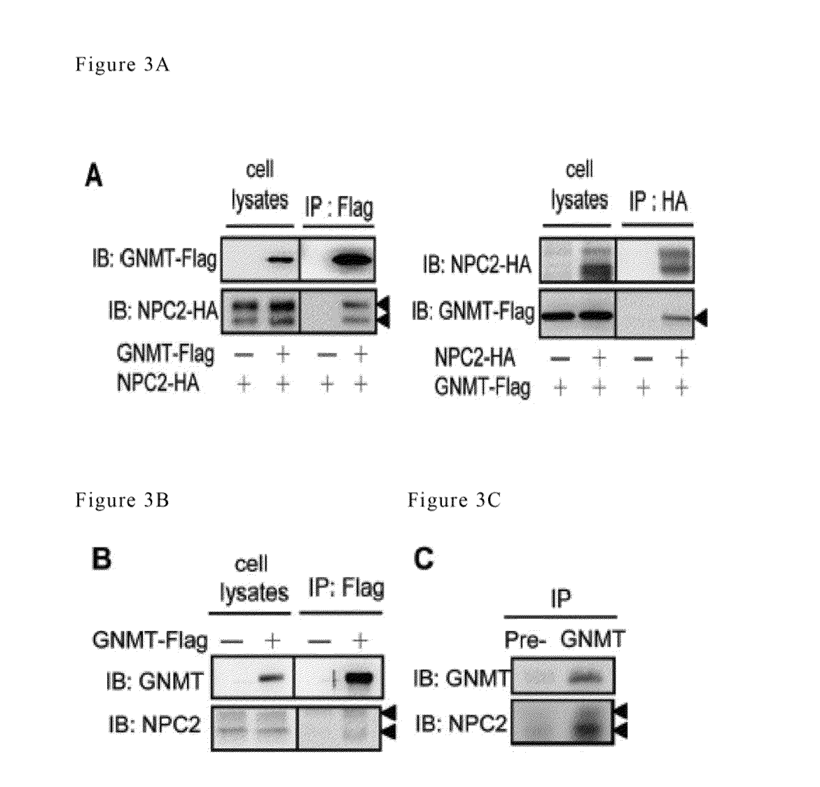

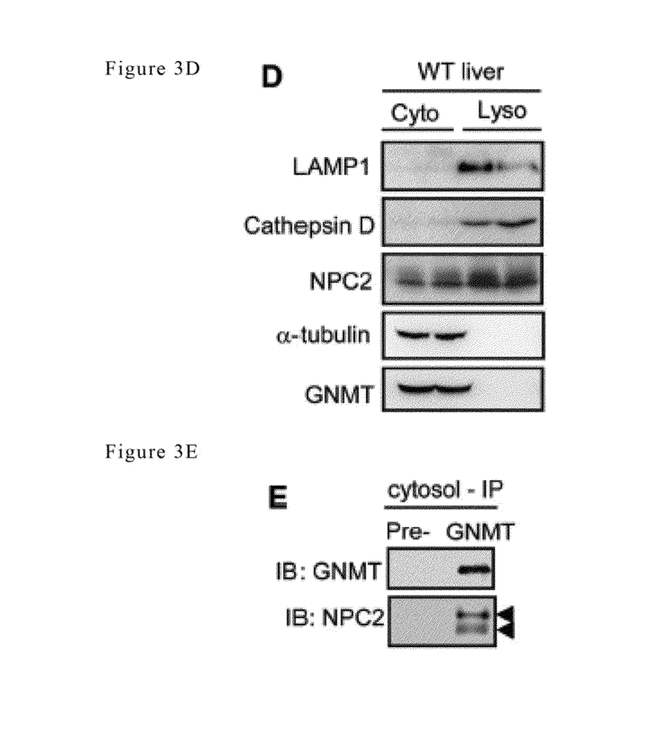

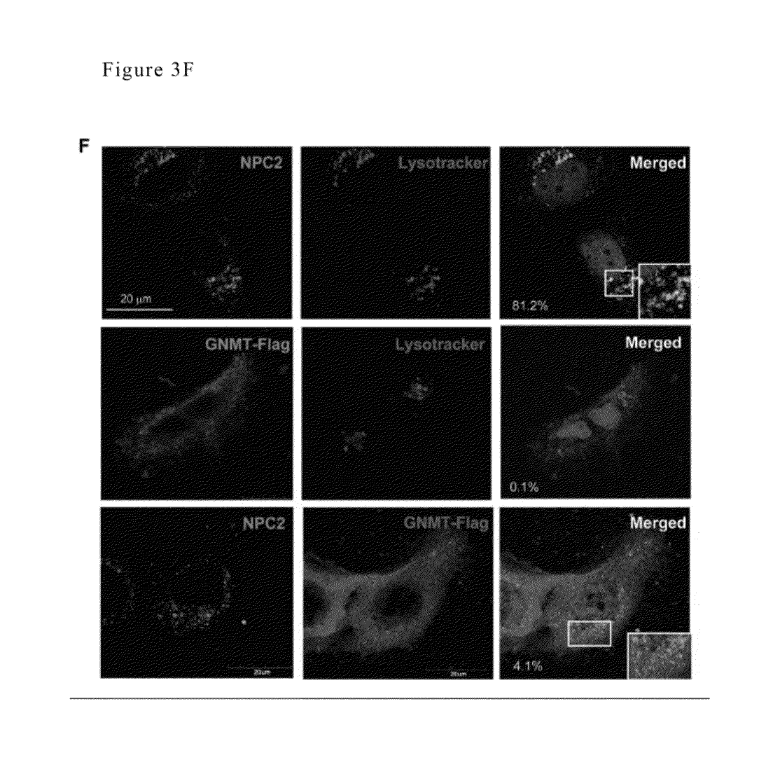

293T cells were cotransfected with GNMT-Flag and NPC2-HA for 24 h and harvested for commmunoprecipitation. GNMT was precipitated by anti-Flag beads and immunoblotted with anti-HA to detect NPC2 expression (arrowhead). NPC2 was precipitated by anti-HA beads and immunoblotted with anti-Flag to detect GNMT expression (arrowhead). As shown in FIG. 3A, both GNMT and NPC2 proteins were capable of binding with their counterpart proteins in coimmunoprecipitation experiments using anti-HA or anti-Flag tag antibodies. Consistent with the above-described overexpression system, endogenous NPC2 was detected in immunoprecipitates prepared from the SK-Hep1 (hepatoma cell line) transfected with pGNMT-Flag (FIG. 3B) and WT mouse liver lysates (FIG. 3C).

To prove GNMT colocalization and interaction with NPC2 in the cytosol, we isolated various lysosomal and cytosolic fractions from WT mouse liver for Western blot and communoprecipitation analyses. As a result, approximately 80% of the NPC2 was expressed in lysosomes (indicated by the lysosomal markers LAMP 1 and cathepsin D), and 20% was expressed in the cytosol. In contrast, GNMT was expressed in the cytosol (FIG. 3D).

Liver cytosol fractions from WT mice were precipitated with anti-GNMT antibodies and immunoblotted with anti-NPC2 antibodies to detect endogenous NPC2 expression, and to confirm GNMT-NPC2 interaction within the cytosol (FIG. 3E). To gain further insight into GNMT-NPC2 interaction, we used immunofluorescence to assess GNMT and NPC2 colocalization. HuH7 cells were transfected with GNMT-Flag and fixed for immunofluorescence assays using antibodies against Flag, NPC2, and Lysotracker to detect colocalization among GNMT, NPC2, and lysosomes. The data indicated that 81.2% of endogenous NPC2 colocalized with Lysotracker in punctate structures, but part of the diffuse perinuclear compartment of NPC2 did not (FIG. 3F, upper panel inset). GNMT-Flag did not colocalize with Lysotracker (FIG. 3F, middle panel). It is important to note that a small amount (4.1%) of diffused NPC2 colocalized with GNMT-Flag in the cytosol of HuH7 cells (FIG. 3F, lower panel inset), suggesting that GNMT-NPC2 interaction did not take place in the lysosome.

To map GNMT-NPC2 interactive domains, we constructed nine plasmids containing different regions of either GNMT or NPC2. The results indicate that the amino-terminal half of GNMT was not capable of pulling down NPC2, but the carboxyl-terminal half (containing amino acid residues 171-295) of GNMT was capable. On the other hand, the C region of NPC2 (containing amino acid residues 81-105) was the primary domain for GNMT interaction.

GNMT Enhances NPC2 Protein Stability

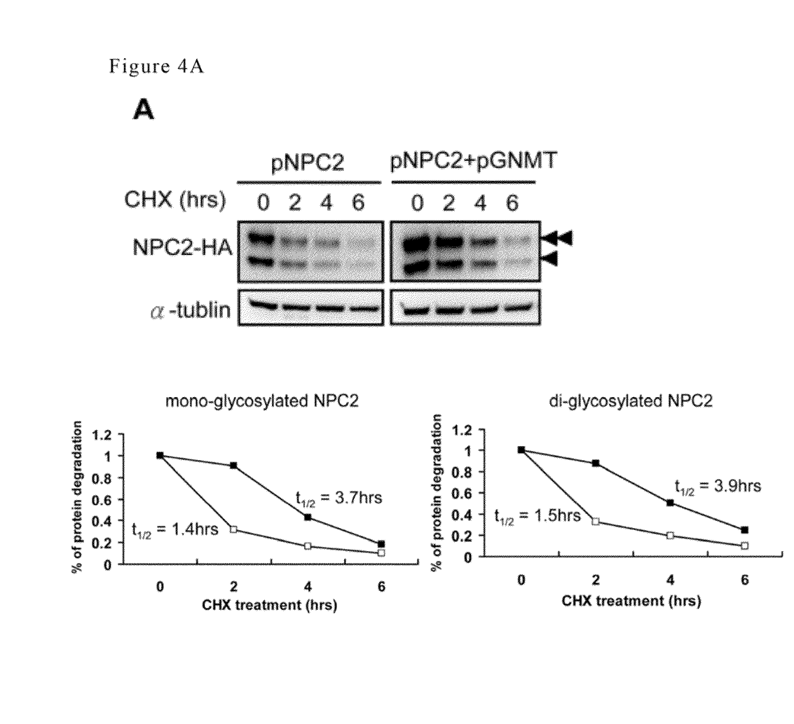

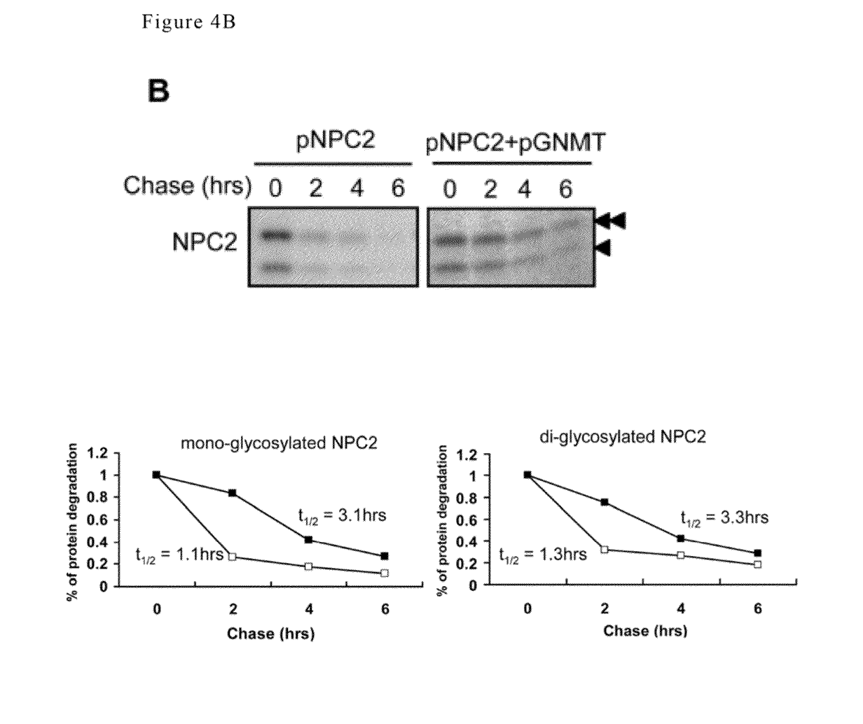

Because the protein level of NPC2 decreased significantly in Gnmt−/− mice liver tissues while mRNA level remained unchanged (FIGS. 2A, B), we assumed that GNMT positively affects NPC2 stability at the posttranslational level. In cells transfected with pNPC2-HA, cycloheximide treatment degraded NPC2 isoforms in a time-dependent manner, with half-lives of approximately 1.4-1.5 h (FIG. 4A). When cells were cotransfected with pNPC2-HA and pGNMT-Flag, the half-lives of mono-glycosylated and diglycosylated NPC2 isoforms increased to 3.7 and 3.9 h, respectively (see FIG. 4A). Pulse-chase experiments were performed to confirm these findings. In the absence of GNMT, the half-lives of NPC2 isoforms were 1.1 and 1.3 h, respectively (FIG. 4B). In the presence of GNMT, the half-lives of NPC2 isoforms more than doubled to 3.1 and 3.3 h, respectively (see FIG. 4B). Taken together, GNMT over-expression doubled the half-lives of NPC2 isoforms in cells.

GNMT-NPC2 Influences Intracellular Cholesterol Homeostasis

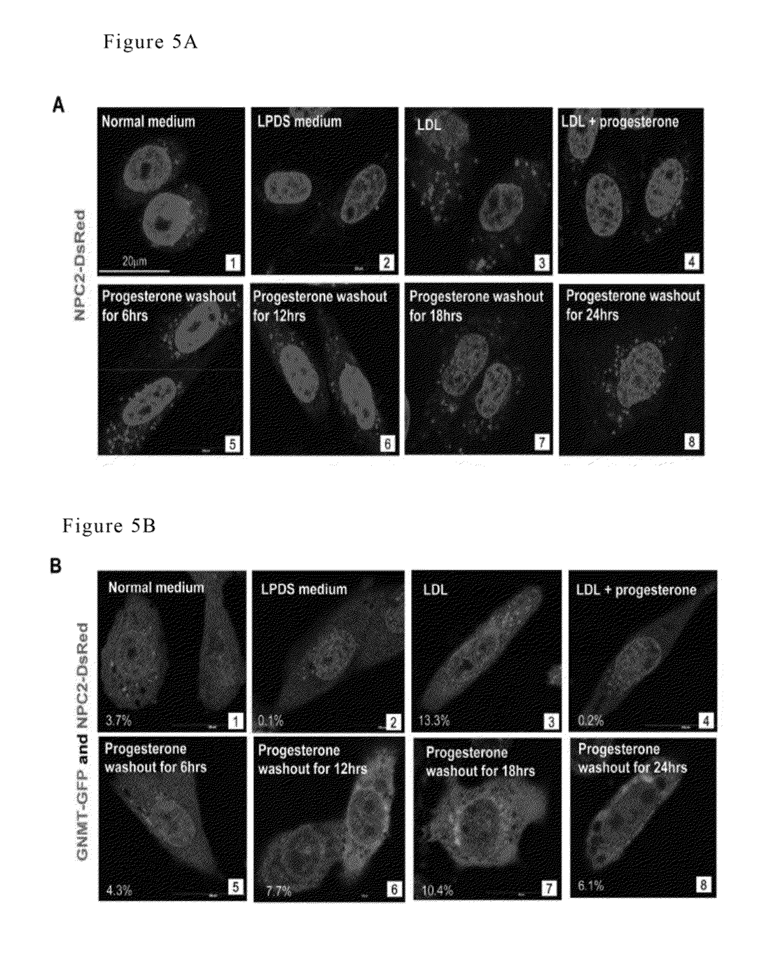

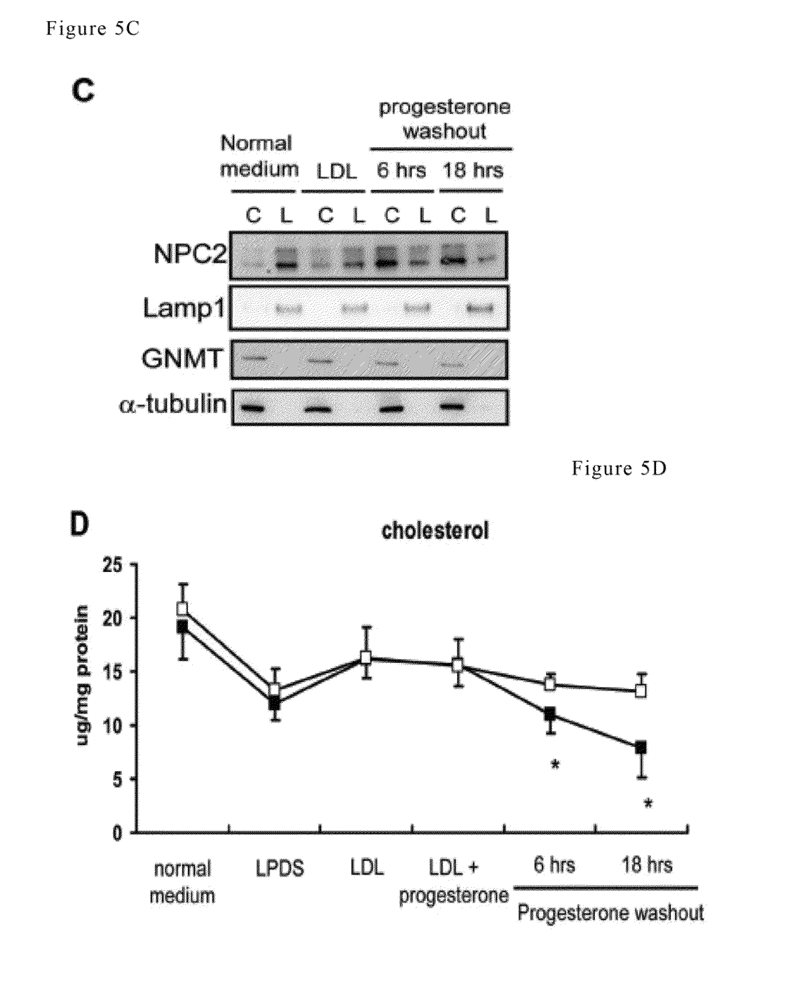

To monitor dynamic distribution and colocalization between GNMT and NPC2, we used immunofluorescent staining, confocal microscopy and Western blot to detect NPC2-GNMT colocalization in cells treated with LDL and progesterone. Progesterone inhibits cholesteryl ester synthesis and blocks cholesterol trafficking pathways. When CHO cells were transfected with NPC2-DsRed and treated with LDL and progesterone for 24 h, NPC2 was accumulated in lysosomes (FIG. 5A-4) because of the inhibitory effect of progesterone on cholesteryl ester synthesis in the endoplasmic reticulum. NPC2 subsequently appeared in the cytosol, peaking between 6 and 18 h after progesterone was removed front the medium (FIG. 5A-5-7).

When CHO cells overexpressed GNMT-GFP and NPC2-DsRed, colocalization signals between GNMT and NPC2 became prominent in the cytosol between 6 and 18 h after the removal of progesterone (FIG. 5B-5-7). According to cytosol-lysosome isolation results, endogenous NPC2 was predominantly expressed in the lysosomal fractions of SK-Hep1 cells before LDL and progesterone treatment (FIG. 5C). Endogenous NPC2 was also increased in cytosolic fractions after the removal of progesterone from the medium at 6 and 18 h. In contrast, GNMT remained in cytosolic fractions throughout the experiment (see FIG. 5C).

To evaluate whether GNMT coordinates with NPC2 in regulating intracellular homeostasis of cholesterol, SK-Hep1. cells were infected with Ad-GNMT for 24 h before treatment with LDL and progesterone. Compared to cells infected with a vector control, the cholesterol levels in GNMT-overexpressed SK-Hep1 cells decreased significantly 6-18 h after the removal of LDL and progesterone (FIG. 5D, P<0.05), indicating that the presence of GNMT prevents cholesterol accumulation in the cells.

Expression Pattern Of NPC2 Is Dysregulated In Fatty Liver Tissues

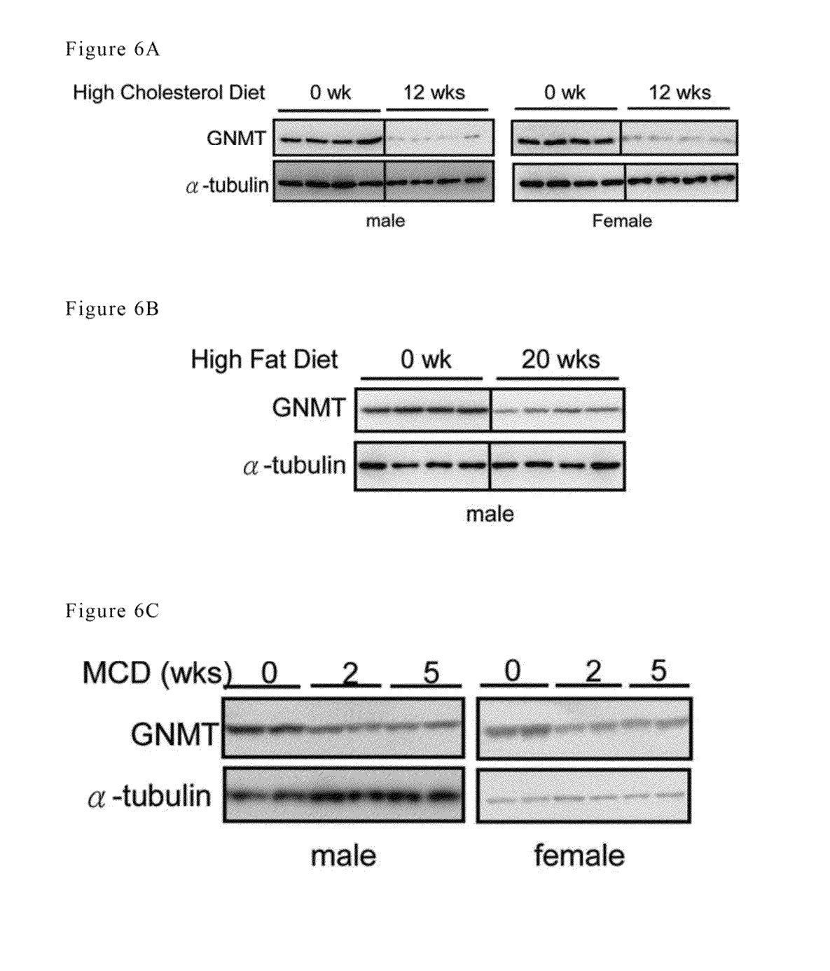

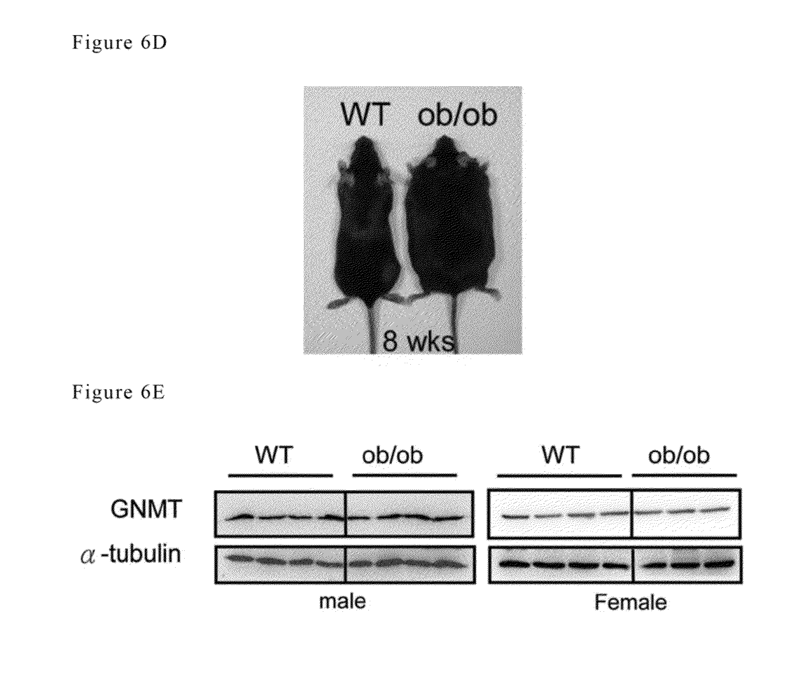

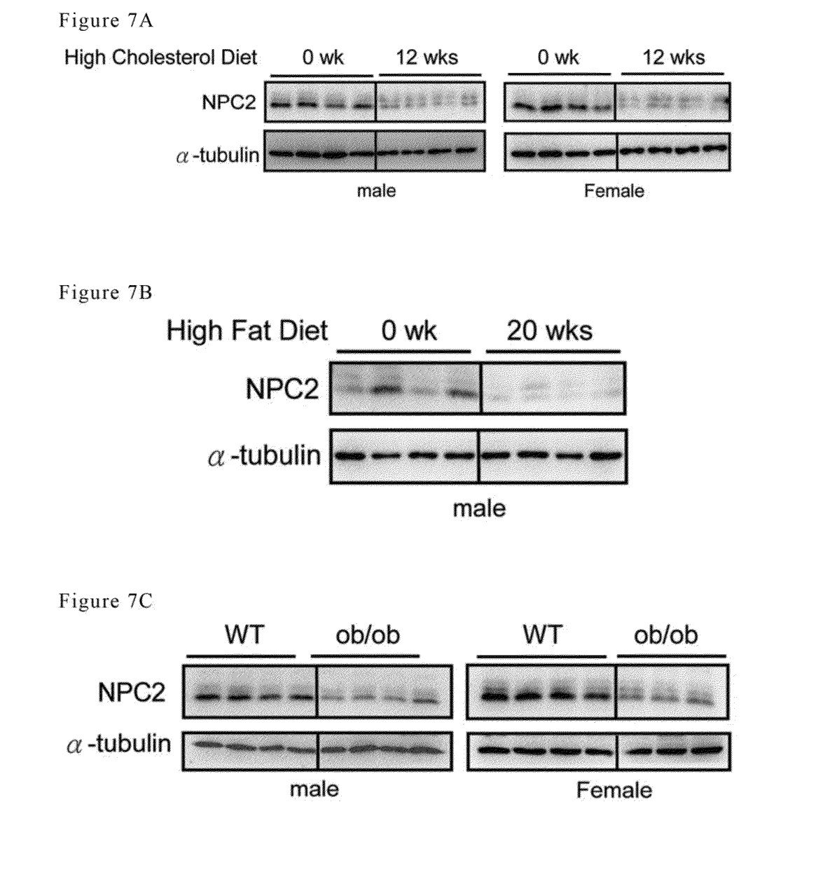

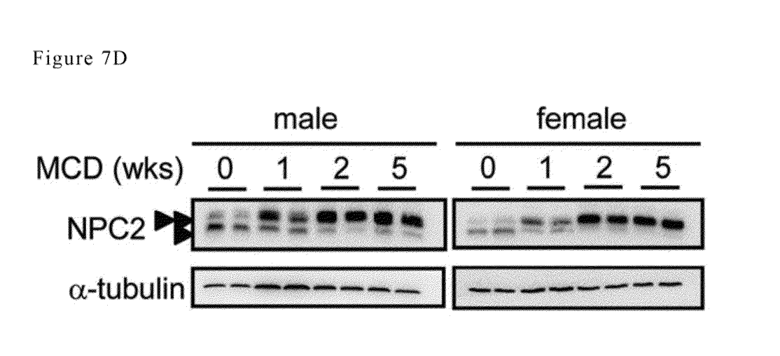

In mice, dietary models (such as MCD, high-cholesterol and high-fat diets) and genetic models (such as ob/ob mice) both mimic NAFLD in humans. We therefore fed WT mice a high-cholesterol diet, high-fat diet or MCD diet to prove the downregulation of GNMT in fatty liver tissues. As expected, we found that GNMT was downregulated in mice fed a high-cholesterol diet, high-fat diet or MCD diet but not in ob/ob mice (FIGS. 6A-E). No changes in GNMT expression were noted in mice fed a normal diet. NPC2 was downregulated in ob/ob mice as well as in mice fed a high-cholesterol diet or high-fat diet (FIGS. 7A-C). However, in the MCD diet-induced steatosis and steatohepatitis mouse model, a progressive increased glycosylated-NPC2 expression was observed in both genders of WT mice liver (FIG. 7D).

No appreciable differences in NgBR expression levels were observed in Gnmt−/− mice. In addition, co-immunoprecipitate GNMT did not observe the expression of NgBR, suggesting that the interaction between GNMT and NPC2 is independent of NgBR. Because NPC2 cholesterol binding occurs at both acidic and neutral pH levels, GNMT may physiologically interact with NPC2 and maintain NPC2 function in the cytosol before NPC2-NgBR interaction. In addition to supporting the idea of GNMT as an NPC2-interacting protein, these findings suggest avenues for enhancing NPC2 protein stability and preventing intracellular cholesterol accumulation.

### Example 3

### GNMT Can Be Used As A Prophylactic Or Therapeutic Agent For Fatty Liver Related Diseases

According to the two-hit hypothesis, lipid accumulation and subsequent inflammation and oxidative stress may trigger liver damage and HCC. In example 1, it is showed that Gnmt^{-/-} mice had signs of hepatic steatosis with increased cholesterol deposition in liver tissues, as well as inflammatory infiltration before HCC formation (FIG. 1). According to our previous report, 50% male and 100% female Gnmt^{-/-} mice developed HCC spontaneously, suggesting that loss of GNMT is associated with gender disparity of liver cancer susceptibility. By this invention, it is important to note that the percentages of female Gnmt^{-/-} mice with fatty change were higher than in male Gnmt^{-/-} mice at 11 weeks and 9 months. In 18- to 24-month tumor-bearing mice, 100% of female Gnmt^{-/-} mice had fatty change. In contrast, only 63% (10/16) of male Gnmt^{-/-} mice had fatty change. This observation implies the possibility that deterioration of lipid metabolism in liver may affect the development of HCC, especially in female Gnmt^{-/-} mice.

This invention firstly discloses the demonstration that GNMT plays an important role in regulating cholesterol homeostasis via interaction with NPC2. GNMT deficiency attenuates NPC2 protein stability and triggers cholesterol accumulation in the liver tissues. Because NASH is one of the key factors involved in cirrhosis and HCC development, data presented in the examples have both physiological and pathological significance. These novel findings may provide important implications regarding the development of diagnostic or therapeutic strategies for fatty liver patients, and possibly for HCC as well.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A pharmaceutical composition for in treating or preventing fatty liver related disease, comprising Glycine N-methyltransferase (GNMT) as active ingredient, and a pharmaceutically acceptable carrier, diluent or excipient.

2. The pharmaceutical composition of claim 1, wherein the fatty liver related disease comprises Non-alcoholic fatty liver diseases (NAFLD).

3. The pharmaceutical composition of claim 1, wherein the fatty liver related disease comprises non-alcoholic steatohepatitis (NASH).

4. The pharmaceutical composition of claim 1, wherein the fatty liver related disease comprises liver cirrhosis.

5. The pharmaceutical composition of claim 1, wherein the fatty liver related disease comprises hepatocellular carcinoma (HCC).

6. The pharmaceutical composition of claim 1, which is used in enhancing the activity and stability of NPC2 protein in liver cells.

7. The pharmaceutical composition of claim 1, which is used in the improvement of GNMT-NPC2 interaction, and further to decrease or prevent cholesterol accumulation in liver cells.

8. A method for diagnosing the occurrence of fatty liver disease in a tested animal, comprising collecting a blood or liver sample from the animal; and detecting the content or expression level of GNMT protein by using a detecting agent.

9. The method of claim 8, wherein the detecting agent for GNMT protein comprises an anti-GNMT antibody.
